(19) United States
(12) Patent Application Publication
(10) Pub. No.: US 2022/0087717 A1
(43) Pub. Date: Mar. 24, 2022

(54) DELIVERY ASSISTANCE TOOL FOR SHOULDER DYSTOCIA

(71) Applicant: Li-Cheng LU, Taoyuan City (TW)

(72) Inventor: Li-Cheng LU, Taoyuan City (TW)

(21) Appl. No.: 17/080,639

(22) Filed: Oct. 26, 2020

(30) Foreign Application Priority Data
Sep. 18, 2020 (TW) 109132317

(51) Int. Cl. A61B 17/42

(52) U.S. Cl. CPC A61B 17/42; A61B 2017/4216

(57) ABSTRACT

The disclosure provides a delivery assistance tool configured to be sleeved on shoulders of a fetus for releasing shoulder dystocia. The delivery assistance tool includes a wrapping part and at least one rope. The wrapping part has two opposite ends fixed to each other so as to form an accommodation space. The accommodation space is configured to accommodate the shoulders, chest, and back of the fetus. The wrapping part has at least one rope channel surrounding the accommodation space. The rope is partially disposed in the rope channel and disposed through two opposite openings of the rope channel so that different parts of the rope are respectively located inside and outside the rope channel. An opening of the accommodation space is adjustable by adjusting the length of the part of the rope in the rope channel.

### CROSS-REFERENCE TO RELATED APPLICATIONS

This non-provisional application claims priority under 35 U.S.C. § 119(a) on Patent Application No(s). 109132317 filed in Taiwan, R.O.C. on Sep. 18, 2020, the entire contents of which are hereby incorporated by reference.

### TECHNICAL FIELD

The disclosure relates to an instrument used for delivery, more particularly to a delivery assistance tool for shoulder dystocia.

### BACKGROUND

Obstructed labour is when the baby does not exit the pelvis during childbirth due to physically blocked. The main causes may include a large or abnormally positioned baby, a small pelvis, and problems with the birth canal. The treatment of obstructed labour may require forceps delivery, cesarean section or vacuum extraction.

In most cases, prolonged obstructed labour may occur due to too large fetal head to pass through the mother's pelvis. However, in another case, the fetal head has been delivered out of the mother, but the fetal body may be stuck in the mother. Generally, such a case may belong to the shoulder dystocia. The shoulder dystocia is an obstetric emergency when, after birth canal delivery of the head, the baby's anterior shoulder gets caught above the mother's pubic bone. When the shoulder dystocia occurs, the upper body of the fetus is stuck so unable to breathe even the head has been delivered, and the umbilical cord is compressed to stop providing oxygen, in such a case, any failure of timely and appropriate action can lead to fetal hypoxia. Initial solutions to release the shoulder typically include pushing on the abdomen above the pubic bone or change the position of the fetal shoulder, but none of them is reliable and sometimes still requires invasive approach or surgery, increasing the risk to the maternal and the fetus.

### SUMMARY OF THE INVENTION

The disclosure provides a delivery assistance tool that can help the doctor or nurse to release a shoulder dystocia during delivery.

One embodiment of the disclosure provides a delivery assistance tool configured to be sleeved on shoulders of a fetus for releasing shoulder dystocia. The delivery assistance tool includes a wrapping part and at least one rope. The wrapping part has two opposite ends fixed to each other so as to form an accommodation space. The accommodation space is configured to accommodate the shoulders, chest, and back of the fetus. The wrapping part has at least one rope channel, and the rope channel surrounds the accommodation space, such that the rope channel surrounds the shoulders, chest, and back of the fetus when the accommodation space accommodates them. The rope is partially disposed in the rope channel and disposed through two opposite openings of the rope channel so that different parts of the rope are respectively located inside and outside the rope channel. An opening of the accommodation space is adjustable by adjusting the length of the part of the rope in the rope channel, such that the wrapping part can compress the shoulders of the fetus to shorten the distance between them, thereby releasing the shoulder dystocia.

According to the delivery assistance tool as discussed above, by pulling the ropes outwards and squeezing the wrapping part, the accommodation space of the wrapping part can be shrunk, such that the wrapping part can compress the shoulders of the fetus and thus shortening the distance between the shoulders for releasing the shoulders of the fetus from the pubic bone of the maternal. Therefore, the shoulders of the fetus can smoothly pass through the birth canal of the maternal so as to be delivered out of the maternal. As a result, the delivery assistance tool can help the doctor and the nurse to release the shoulder dystocia of the fetus.

### BRIEF DESCRIPTION OF THE DRAWINGS

The present disclosure will become better understood from the detailed description given herein below and the accompanying drawings which are given by way of illustration only and thus are not intending to limit the present disclosure and wherein:

FIG. 1 is a perspective view of a delivery assistance tool for shoulder dystocia according to one embodiment of the disclosure;

FIG. 2 is another perspective view of the delivery assistance tool in FIG. 1;

FIG. 3 is a schematic view showing a fetus during delivery;

FIG. 4 is a schematic view showing that the delivery assistance tool in FIG. 1 is sleeved on the fetus;

FIG. 5 is a schematic view showing that a wrapping part of the delivery assistance tool in FIG. 4 is shrinking;

FIG. 6 is a schematic view showing that the shoulder of the fetus passes through the birth canal with the help of the delivery assistance tool; and

FIG. 7 is a partial perspective view of a delivery assistance tool for shoulder dystocia according to another embodiment of the disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In addition, the terms used in the present disclosure, such as technical and scientific terms, have its own meanings and can be comprehended by those skilled in the art, unless the terms are additionally defined in the present disclosure. That is, the terms used in the following paragraphs should be read on the meaning commonly used in the related fields and will not be overly explained, unless the terms have a specific meaning in the present disclosure.

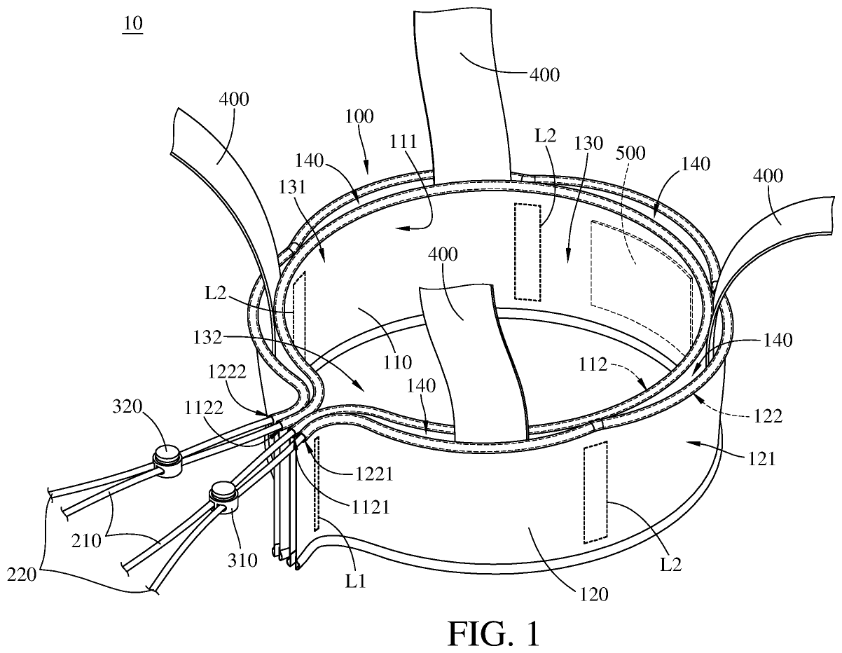

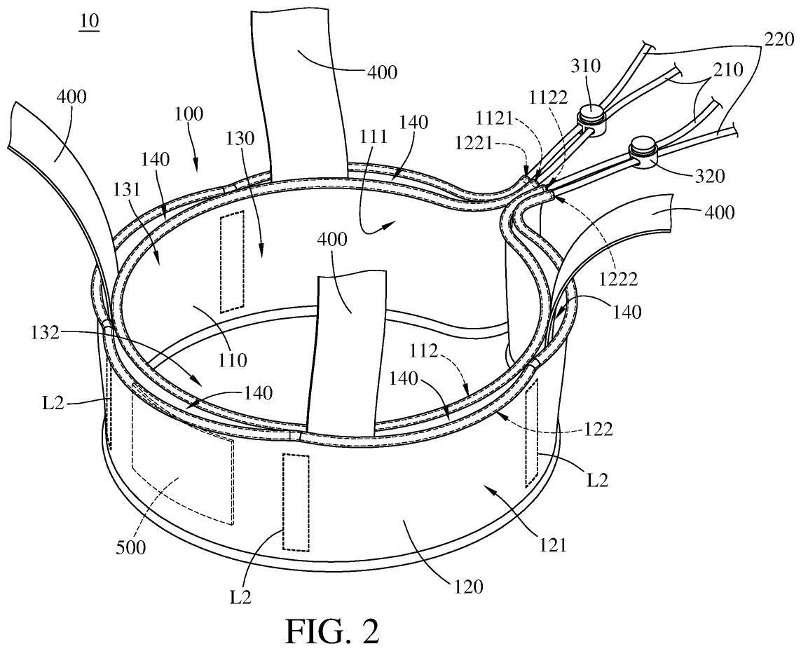

Referring to FIGS. 1 and 2, there are shown a perspective view of a delivery assistance tool 10 for shoulder dystocia according to one embodiment of the disclosure and another perspective view of the delivery assistance tool 10 in FIG. 1.

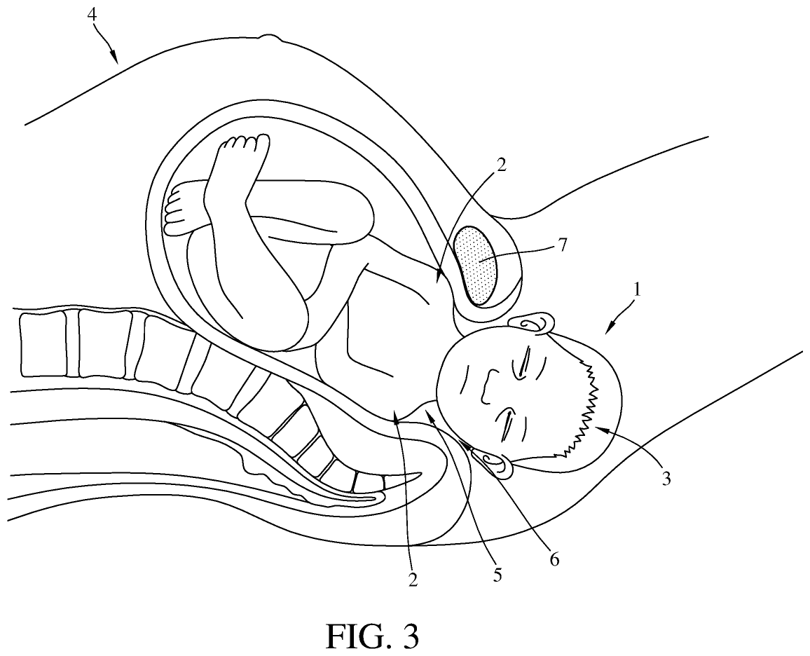

In this embodiment, the delivery assistance tool 10 is configured to help the baby's shoulders pass through the birth canal during delivery so as to release a shoulder dystocia. As the fetus 1 and shoulders 2 shown in FIG. 3, the delivery assistance tool 10 can be sleeved on shoulders 2 of a fetus 1.

In detail, in this embodiment, the delivery assistance tool 10 includes a wrapping part 100 and two ropes 210 and 220. In this or another embodiment, the delivery assistance tool 10 may include two fasteners 310 and 320, a plurality of handles 400, and a reinforcement component 500.

The wrapping part 100 includes an inner layer 110 and an outer layer 120. Two opposite ends of the inner layer 110 and two opposite ends of the outer layer 120 are sutured to each other via, for example, a suture L1. The inner layer 110 is surrounded by the outer layer 120, and the inner layer 110 surrounds and defines an accommodation space 130. The accommodation space 130 is sized to accommodate the upper body of the fetus 1 (e.g., the shoulder 2, chest, and back of the fetus 1). The outer layer 120 is located at the side of the inner layer 110 opposite to the accommodation space 130. The inner layer 110 has a first surface 111 facing the accommodation space 130, and the outer layer 120 has a second surface 121 facing away from the first surface 111. The first surface 111 has a friction coefficient larger than that of the second surface 121; that is, the roughness of the first surface 111 of the inner layer 110 is greater than that of the second surface 121 of the outer layer 120. In this embodiment, the first surface 111 of the inner layer 110 may have artificial short fur thereon or may has a rubbery texture.

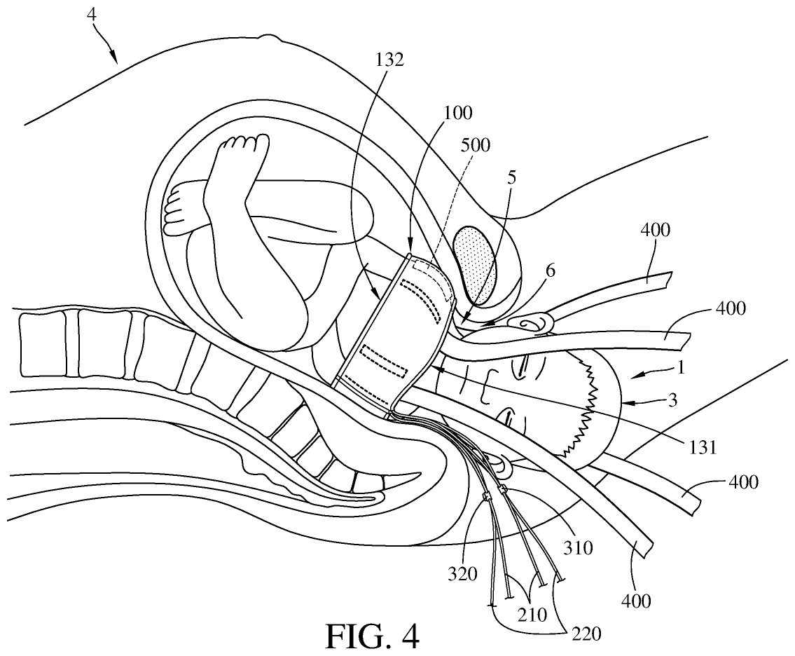

In this embodiment, the wrapping part 100 opens at two opposite sides, such as a first opening 131 and a second opening 132 of the accommodation space 130. When the wrapping part 100 is sleeved on the shoulders 2 of the fetus 1 in the maternal 4, the first opening 131 of the accommodation space 130 is located closer to the birth canal orifice 6 than the second opening 132 (as shown in FIG. 4). The inner layer 110 further has a rope channel 112, and the outer layer 120 further has a rope channel 122. The rope channel 112 of the inner layer 110 and the rope channel 122 of the outer layer 120 both are located aside the first opening 131 of the accommodation space 130 and surround the accommodation space 130. The rope channel 112 of the inner layer 110 may be formed by folding an edge of the inner layer 110 and then suturing it to the inner layer 110. Similarly, the rope channel 122 of the outer layer 120 may be formed by folding an edge of the outer layer 120 and then suturing it outer layer 120.

The rope channel 112 of the inner layer 110 has two openings 1121 and 1122 located opposite to each other, and the rope channel 122 of the outer layer 120 has two openings 1221 and 1222 located opposite to each other. The parts, where the inner layer 110 and the outer layer 120 are sutured to each other, are located adjacent to the openings 1121 and 1122 of the rope channel 112 and the openings 1221 and 1222 of the rope channel 122. The openings 1121 and 1122 of the rope channel 112 is located between the openings 1221 and 1222 of the rope channel 122, and the opening 1121 is located closer to the opening 1221 than the opening 1122. At least part of the rope 210 is disposed through the openings 1121 and 1122 of the rope channel 112, and the rest part of the rope 210 is not in the rope channel 112. At least part of the rope 220 is disposed through the openings 1221 and 1222 of the rope channel 122, and the rest part of the rope 220 is not in the rope channel 122. The parts of the ropes 210 and 220 that are located outside the rope channels 112 and 122 can be fastened to each other. Specifically, the fasteners 310 and 320 are in a form of any suitable buckle for rope, the parts of ropes 210 and 220 exiting from the openings 1121 and 1221 penetrate through the fastener 310 and may be bundled to each other, and the parts of the ropes 210 and 220 exiting from the openings 1122 and 1222 penetrate through the fastener 310 and may be bundled to each other.

In this embodiment, the parts of the inner layer 110 that forms the accommodation space 130 can be sutured to the outer layer 120 via one or more sutures L2, where the sutures L2 may be spaced apart from each other so that there are a plurality of insertion holes 140 formed between the outer layer 120 and the inner layer 110. The insertion holes 140 extend from the side of the wrapping part 100 close to the first opening 131 towards the side of the wrapping part 100 close to the second opening 132. The insertion holes 140 allows the insertions of fingers.

The handles 400 are connected to the side of the inner layer 110 located close to the first opening 131. Specifically, the handles 400 can be respectively disposed at the insertion holes 140 and stick out from the side of the inner layer 110 located close to the first opening 131 so that the handles 400 are spaced apart from each other and extend outwards in a direction away from the second opening 132. Each of the handles 400 is sized to be handheldable. When the wrapping part 100 is sleeved on the fetus 1, the wrapping part 100 can be pulled or rotated by pulling the handles 400. The outward movement or rotational movement of the wrapping part 100 can cause the fetus 1 to move or rotate in the required position. In this disclosure, the handles 400 are not restricted to be connected the inner layer 110; in some other embodiments, the handles may be connected to the outer layer. In addition, the quantity of the handles is not restricted to be four as shown in FIG. 2; in some other embodiments, the delivery assistance tool may include more or less handles.

The reinforcement component 500 is, for example, a plastic sheet. The reinforcement component 500 is disposed on the wrapping part 100 and located between the inner layer 110 and the outer layer 120. The rigidity of the reinforcement component 500 is greater than that of the wrapping part 100. The reinforcement component 500 is configured to provide support to the fetus 1 while using the delivery assistance tool 10.

Referring to FIG. 3, there is shown a schematic view showing the fetus 1 during a delivery. As shown in FIG. 3, a shoulder dystocia occurs, when the shoulders 2 of the fetus 1 get caught by the pubic bone 7 after birth canal delivery of the head 3. The following paragraphs will describe how to use the delivery assistance tool 10 to release the shoulder dystocia.

Referring to FIGS. 2 and 4 to 6, there are shown a schematic view showing that the delivery assistance tool 10 in FIG. 1 is sleeved on the fetus 1, a schematic view showing that the wrapping part 100 of the delivery assistance tool 10 in FIG. 4 is shrinking, and a schematic view showing that the shoulders 2 of the fetus 1 passes through the birth canal 5 with the help of the delivery assistance tool 10.

The first step is to sleeve the wrapping part 100 through the head 3 and all the way down to the shoulders 2. This process can be done by inserting fingers into the insertion holes 140 to adjust the positions of the wrapping part 100 in the birth canal 5. By doing so, the wrapping part 100 will be sleeved on the shoulders 2, chest, and back of the fetus 1 as shown in FIG. 4, and the wrapping part 100 is in the position ready for pulling. At this moment, the parts of the ropes 210 and 220 and the fasteners 310 and 320 are located outside the birth canal 5, and the reinforcement component 500 can provide support to one of the shoulders 2 of the fetus 1.

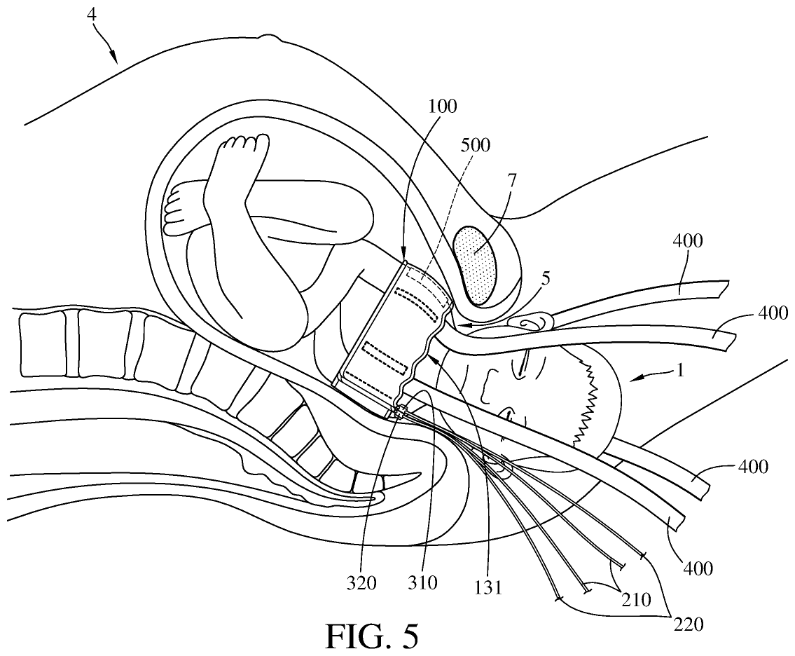

Then, as shown in FIG. 5, the doctor can begin to pull the ropes 210 and 220 outwards while slide the fasteners 310 and 320 to squeeze the wrapping part 100 to shrink the first opening 131 of the accommodation space 130. During this process, the wrapping part 100 and the reinforcement component 500 will slightly compress the shoulders 2 of the fetus 1 to release the shoulders 2 from the pubic bone 7.

During the shrinkage of the first opening 131 of the accommodation space 130, the reinforcement component 500 can provide support to the shoulder 2 of the fetus 1, which may help to compress the shoulders 2 of the fetus 1 so as to release the shoulders 2 of the fetus 1 from the pubic bone 7.

Note that the position of the reinforcement component 500 can be modified as will. In some other embodiments, the reinforcement component 500 may be disposed on the first surface 111 of the inner layer 110 or the outer surface 121 of the outer layer 120. In another embodiment, the delivery assistance tool may omit the reinforcement component 500.

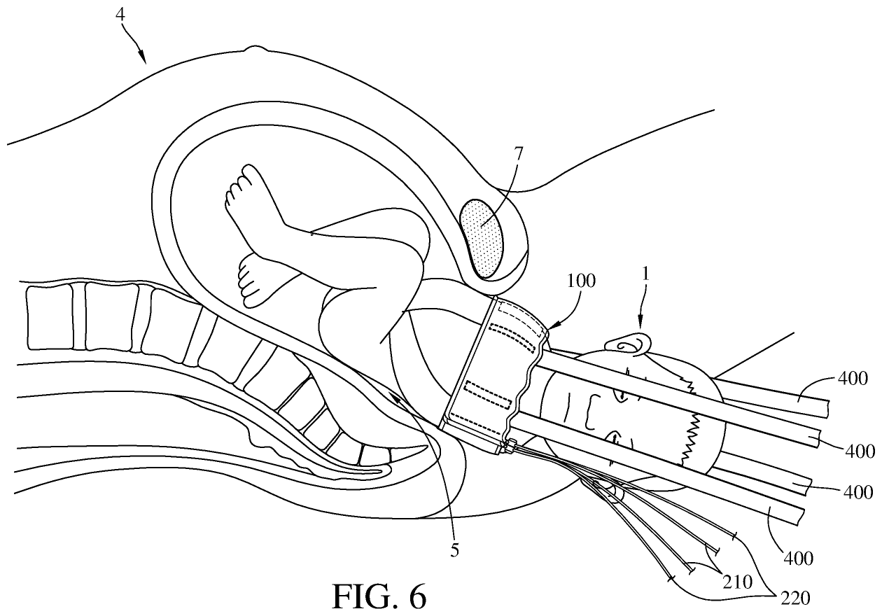

Then, as shown in FIG. 6, the handles 400 is pulled outwards, such that the wrapping part 100 sleeved on the fetus 1 moves the fetus 1 to make the shoulder 2 of the fetus 1 pass through the birth canal 5, thereby releasing the shoulder dystocia.

In this embodiment, by pulling the ropes 210 and 220 outwards and squeezing the wrapping part 100, the accommodation space 130 of the wrapping part 100 can be shrunk, such that the wrapping part 100 can compress the shoulders 2 of the fetus 1 and thus shortening the distance between the shoulders 2 for releasing the shoulders 2 of the fetus 1 from the pubic bone 7 of the maternal 4. Therefore, the shoulders 2 of the fetus 1 can smoothly pass through the birth canal 5 of the maternal 4 so as to be delivered out of the maternal 4. As a result, the delivery assistance tool 10 can help the doctor and the nurse to release the shoulder dystocia of the fetus 1.

In addition, when the wrapping part 100 and the reinforcement component 500 hold the shoulders 2 of the fetus 1, the fetus 1 can be rotated with the wrapping part 100 by manipulating the handles 400. This may help release the shoulder 2 from the pubic bone 7. However, the handles 400 of the delivery assistance tool 10 may be optional. In some other embodiments, the delivery assistance tool may omit the handles 400; in such a casing, the position of the fetus still can be changed by hands.

In addition, the roughness difference between the first surface 111 of the inner layer 110 and the second surface 121 of the outer layer 120 ensures that the fetus 1 can be moved with the wrapping part 100 while pulling the wrapping part 100 outwards. Also, the friction between the wrapping part 100 and birth canal 5 can be small for facilitation of pulling the wrapping part 100 out of the birth canal 5.

In this embodiment, the wrapping part 100 is formed by two stripes of fabric, but the present disclosure is not limited thereto; in some other embodiments, the wrapping part may be formed by a single stripe of fabric folded in half.

In addition, the quantities of the rope channels 112 and 122, the ropes 210 and 220, and the fasteners 310 and 320 can be modified as will; in some other embodiments, there are only one rope channel, one rope, and one fastener in the delivery assistance tool; in another embodiment, the delivery assistance tool may omit the fastener; in such a case, the sized of the first opening can be adjusted by hands.

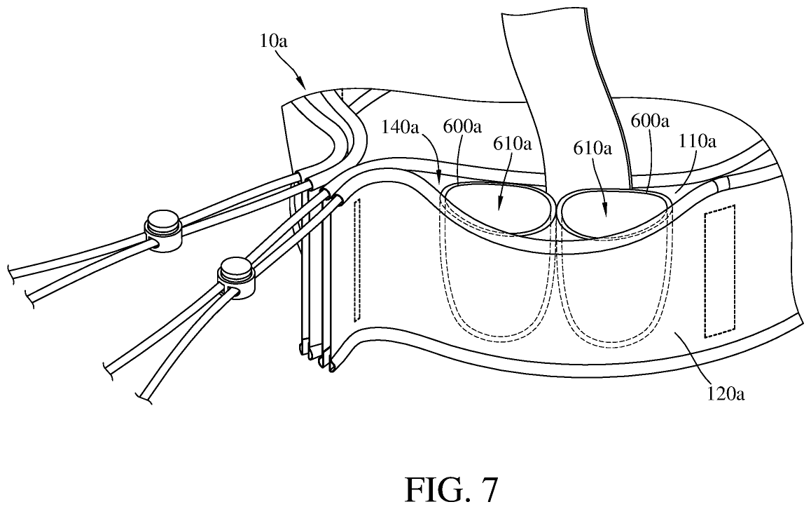

Then, referring to FIG. 7, there is shown a partial perspective view of a delivery assistance tool 10a for shoulder dystocia according to another embodiment of the disclosure.

In this embodiment, the delivery assistance tool 10a is similar to the delivery assistance tool 10 shown in FIG. 1, therefore the following paragraphs will introduce the main differences between them, the similar or same parts will not be described hereinafter.

In this embodiment, the delivery assistance tool 10a further includes a plurality of accommodation components 600a. The accommodation components 600a are, for example, made of soft material. The accommodation components 600a are respectively disposed in the insertion holes 140a between the inner layer 110a and the outer layer 120a. Each of the accommodation components 600a has an insertion hole 610a. The insertion holes 610a allow the insertions of the fingers, thereby facilitating the placement of the delivery assistance tool 10a into the birth canal 5.

Note that the delivery assistance tool of the disclosure is not restricted to be sleeved on the shoulders 2, the chest, and the back of the fetus 1; in some other embodiment, the delivery assistance tool may be sleeved on one shoulder 2, an armpit located away from that shoulder 2, the chest, and the back of the fetus 1. For example, two opposite ends of the wrapping part may not be fixed to each other, before the wrapping part is sleeved on the fetus 1. The wrapping part is sleeved on the fetus by wrapping the wrapping part on the shoulder located close to the pubic bone 7, the chest, the armpit located away from the pubic bone 7 then fixing two opposite ends of the wrapping part to each other. The opposite ends of the wrapping part may be fixed to each other via buttons, Velcro, and so on. As such, when pulling the wrapping part outwards the birth canal 5, the wrapping part is not be easily detached from the fetus 1, since a part of the wrapping part is located at the armpit, thereby facilitating the delivery of the fetus 1.

According to the delivery assistance tools as discussed above, by pulling the ropes outwards and squeezing the wrapping part, the accommodation space of the wrapping part can be shrunk, such that the wrapping part can compress the shoulders of the fetus and thus shortening the distance between the shoulders for releasing the shoulders of the fetus from the pubic bone of the maternal. Therefore, the shoulders of the fetus can smoothly pass through the birth canal of the maternal so as to be delivered out of the maternal. As a result, the delivery assistance tool can help the doctor and the nurse to release the shoulder dystocia of the fetus.

It will be apparent to those skilled in the art that various modifications and variations can be made to the present disclosure. It is intended that the specification and examples be considered as exemplary embodiments only, with a scope of the disclosure being indicated by the following claims and their equivalents.

What is claimed is:

1. A delivery assistance tool, configured to be sleeved on a fetus for releasing shoulder dystocia, comprising:
a wrapping part, wherein the wrapping part has two opposite ends fixed to each other so as to form an accommodation space, the accommodation space is configured to accommodate the fetus, the wrapping part has at least one rope channel, and the at least one rope channel surrounds the accommodation space; and
at least one rope, partially disposed in the at least one rope channel and disposed through two opposite openings of the at least one rope channel so that different parts of the at least one rope are respectively located inside and outside the rope channel;
wherein an opening of the accommodation space is adjustable by adjusting the length of the part of the at least one rope in the at least one rope channel.

2. The delivery assistance tool according to claim 1, wherein the wrapping part has a first surface and a second surface, the first surface faces the accommodation space, the second surface faces away from the first surface, and the friction coefficient of the first surface is larger than the friction coefficient of the second surface.

3. The delivery assistance tool according to claim 1, wherein the accommodation space has a first opening and a second opening opposite to each other, the at least one rope channel is located aside the first opening, the wrapping part has a plurality of insertion holes spaced apart from each other, and the plurality of insertion holes extend from a side of the wrapping part close to the first opening towards a side of the wrapping part close to the second opening.

4. The delivery assistance tool according to claim 3, further comprising a plurality of accommodation components, wherein the plurality of accommodation components are respectively disposed in the plurality of the insertion holes.

5. The delivery assistance tool according to claim 1, further comprising a fastener, wherein the fastener is disposed on the part of the at least one rope located outside the at least one rope channel, the fastener is configured to squeeze the wrapping part so as to adjust the opening of the accommodation space.

6. The delivery assistance tool according to claim 1, further comprising a plurality of handles, wherein the accommodation space has a first opening and a second opening opposite to each other, the at least one rope channel is located aside the first opening, the plurality of handles are spaced apart from each other and connected to a side of the wrapping part close to the first opening, and the plurality of handles extends outwards in a direction away from the second opening.

7. The delivery assistance tool according to claim 1, further comprising a reinforcement component, wherein the reinforcement component is disposed in the wrapping part, the rigidity of the reinforcement component is greater than the rigidity of the wrapping part, and the reinforcement component is configured to provide support to the fetus.

8. The delivery assistance tool according to claim 1, wherein the wrapping part comprises an inner layer and an outer layer, two opposite ends of the inner layer and two opposite ends of the outer layer are fixed to each other, the outer layer surrounds the inner layer, the inner layer surrounds the accommodation space, the quantities of the at least one rope channel and the at least one rope both are two, the two rope channels are respectively located at the inner layer and the outer layer, and the two ropes are respectively and partially disposed in the two rope channels and disposed through opposite openings of the two rope channels so that different parts of the two ropes are respectively located inside and outside the two rope channels.

9. The delivery assistance tool according to claim 8, wherein the inner layer has a first surface facing the accommodation space, the outer layer has a second surface facing away from the first surface, and the friction coefficient of the first surface is larger than the friction coefficient of the second surface.

10. The delivery assistance tool according to claim 8, wherein the accommodation space has a first opening and a second opening opposite to each other, the two rope channels are located aside the first opening, the inner layer and the outer layer are partially fixed to each other so as to form a plurality of insertion hole spaced apart from each other, and the plurality of insertion holes extend from a side of the wrapping part close to the first opening towards a side of the wrapping part close to the second opening.

11. The delivery assistance tool according to claim 8, further comprising two fasteners, wherein the two fasteners are respectively disposed on the parts of the two ropes located outside the two rope channels, and the fasteners are configured to squeeze the wrapping part so as to shrink the opening of the accommodation space.
